(19) United States
(12) Patent Application Publication
(10) Pub. No.: US 2008/0228074 A1
(43) Pub. Date: Sep. 18, 2008

(54) SYSTEM AND METHOD FOR MEASURING ACOUSTIC PRESSURE AT MULTIPLE LOCATIONS SIMULTANEOUSLY

(76) Inventor: Jeffrey A. Ketterling, New York, NY (US)

Correspondence Address: KEITH D. NOWAK, CARTER LEDYARD & MILBURN LLP, 2 WALL STREET, NEW YORK, NY, 10005, US

(21) Appl. No.: 12/046,258

(22) Filed: Mar. 11, 2008

Related U.S. Application Data
(60) Provisional application No. 60/894,314, filed on Mar. 12, 2007.

(51) Int. Cl. A61B 8/00; H04R 31/00

(52) U.S. Cl. 600/438; 29/594

(57) ABSTRACT

The present invention provides a system and method for measuring acoustic pressure at multiple locations simultaneously. An array transducer device is prepared where a polyimide film is bonded to a piezopolymer membrane using a non-conductive epoxy. The polyimide is etched with a linear array pattern having array elements, connection pads and electrical connections connecting the array elements to the connection pads. The array pattern is electronically accessed by linking traces of a customized printed circuit board to the electrical connections of the array pattern via ZIF connectors.

### PRIORITY AND RELATED APPLICATION

This application claims priority to U.S. Provisional Patent Application No. 60/894,314, filed Mar. 12, 2007, entitled “System and Method for Measuring Acoustic Pressure at Multiple Locations Simultaneously,” which is hereby incorporated by reference in its entirety.

### FIELD OF THE INVENTION

The present invention relates to systems and methods for measuring acoustic pressure. In particular, the present invention relates to a system and method for taking measurements of focused shock waves in an acoustic field at multiple locations simultaneously.

### BACKGROUND OF THE INVENTION

Ultrasound is used in medicine for therapeutic and surgical applications. For example, High Intensity Focused Ultrasound (HIFU) can be used for tissue ablation. For surgical applications, bursts of focused ultrasound energy three orders of magnitude more intense than diagnostic ultrasound are emerging as a noninvasive option for treating cancer and other medical procedures. Ultrasound therapy is also commonly used to pulverize kidney stones and gallstones. Lithotripsy, specifically Shock Wave Lithotripsy (SWL), is a primary non-surgical method for treating patients with large kidney stones. SWL uses lithotripters to emit high-pressure, focused shock waves to break kidney stones into smaller fragments that then pass out of the body through the urinary tract.

The original lithotripter used in the field is a Domier HM3 lithotripter. The HM3 emitted low pressure shockwaves with a wide focal spot. Despite the effectiveness of the HM3 lithotripters, its use caused harm to renal and surrounding tissues. As a means of “improving” lithotripters and reducing the harm to tissues, manufacturers increased peak pressure and reduced the focal zone in newer model lithotripters. These “improvements” were based on the assumption that higher pressure will more effectively fragment a stone and smaller focal sizes will permit more accurate targeting of stones thereby reducing the chance of tissue trauma. However, the newer model lithotripters do not fragment stones any better than older model lithotripters and run the risk of an increased chance of complications.

In order to improve lithotripters there is a need to better understand how lithotripters fragment stones and sometimes damage tissue. That understanding is possible with improved measurement tools that will provide an understanding of lithotripters' acoustic fields, bioeffects and calibration constraints. Such a measuring tool should accurately characterize the intensity or pressure of waves generated in a lithotripter acoustic field. Presently, acoustic field characterization is based on focal beamwidth measurements with a single element hydrophone. The beamwidth measurement is obtained by quantifying a collection of sequential single point (point-by-point) measurements within the focal plane using single-element hydrophones. The accuracy of these measurements is reliable when the pressure field is spatially stable, but unreliable when there are transient spatial variations in the pressure or acoustic field.

For instance, shock variations are present in the acoustic field of a Domier HM3 type of lithotripter device. The exact nature of the beam from shock to shock cannot be resolved with single point measurements. Single point measurements taken with a single element hydrophone only yield statistical information about the spatial distribution of the acoustic field. In addition, use of a single element hydrophone only averages sequential set points leading to an overestimation of beamwidth. Accordingly, there is a need for measurement tools that can quickly and accurately measure instantaneous beamwidths of focused shock waves.

Attention is therefore directed by the present invention to developing techniques for measuring ultrasound pressures at several points simultaneously. The desirable techniques will allow measurement of spatially varying transients. Improved measurement tools for measuring an acoustic field will help determine optimal lithotripter device parameters and ensure accurate lithotripter calibration. A hydrophone is one such measurement tool that, if improved, can aid in improving lithotripsy. Ideally, the hydrophone should measure the acoustic field of the SWL at multiple locations simultaneously, instantaneously and in more than one dimension. Thus, the hydrophone should be able to characterize instantaneous beamwidths regardless of shock variability present in certain acoustic fields. The measurements should express the spatial properties of the acoustic field in the SWL focal zone, such as the location of the peak pressure.

### BRIEF SUMMARY OF THE INVENTION

In accordance with the present invention a system and method for simultaneously measuring acoustic pressure at multiple locations involves providing a linear hydrophone array transducer device having a set of detection elements in one or two dimensions. The measurement method includes: placing the transducer device in the field of a shock wave emitted by a lithotripter and acquiring acoustic field pressure data at multiple locations simultaneously from the emitted shock wave. The method of fabricating the transducer device includes providing a polyimide film bonded to a piezopolymer membrane with epoxy, the membrane and the film being pressed into an assembly where the assembly when placed in an acoustic pressure field obtains signal data at multiple locations from a single shock wave in any dimension. The device may be fabricated using the transducer fabrication principles described in U.S. Patent Publication No. 2005/0264133 corresponding to recently issued patent application Ser. No. 11/136,223, which is incorporated here by reference.

### BRIEF DESCRIPTION OF THE DRAWING

FIG. 1 is a diagram illustrating a one dimensional array pattern of equally spaced elements;

FIG. 2 is a view of an assembled linear hydrophone array transducer device of the present invention;

FIG. 3 is another view of the assembled array transducer device of the present invention;

FIG. 4 is a plan view illustrating electronic access to the transducer through a customized printed circuit board connected to the array pattern of the transducer device of the present invention;

FIG. 5 is a block diagram illustrating a system configuration for the present invention;

FIG. 6 shows a scaled drawing of lithotripter geometries;

FIG. 7 shows a measurement of direct and focal shock waves from a single array element;

FIG. 8 shows normalized element sensitivities; and

FIG. 9 shows beam profiles measured for individual shock waves.

### DETAILED DESCRIPTION OF THE INVENTION

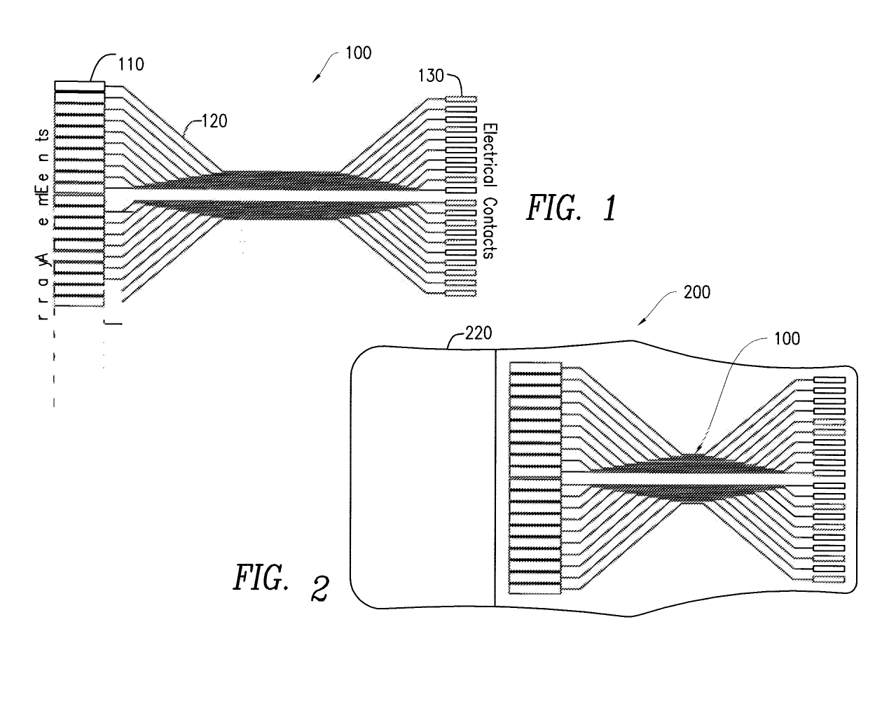

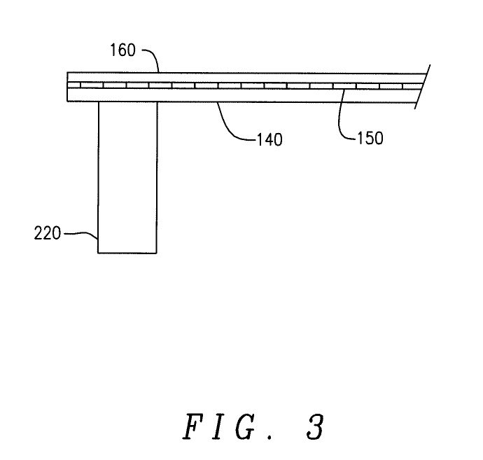

FIGS. 1 and 2 show an exemplary array pattern 100 in a one-dimensional pattern. The array pattern 100 has array elements 110, electrical connection pads 130 and electrical connections 120 linking the elements 110 to the connection pads 130. The array pattern 100 is etched onto a substrate such as a single sided, copper-clad polyimide (CCP) film. A commercially available example of the CCP film is a RFlex 1000L810, manufactured by Rogers Corp. of Chandler, Ariz. CCP film is commonly used to fabricate flex circuits.

In a preferred embodiment, the polyimide film includes a 25-μm thick polyimide layer, a 18-μm thick copper layer and a 20-μm thick adhesive layer bonding the copper to the polyimide. The pattern 100 may be etched onto the flex circuit using techniques known in the art. One preferred method uses standard copper etching techniques. In a preferred embodiment, the positive array image is placed on top of the photoresist coated polyimide and exposed to ultraviolet (UV) light for 2-3 minutes in a UV fluorescent exposure unit, which is commercially available from AmerGraph located in Sparta, N.J. or any equivalent supplier. The polyimide is then transferred to a liquid developer, which removes the photoresist that is exposed to UV light. The developed film is agitated in a ferric chloride bath until all the copper in the areas lacking photoresist are etched away.

The array pattern 100 shows elements 110 being equally spaced apart. The array pattern 100 is linear. There are twenty elements 110 shown, however any number of elements 110 may be employed. The elements 110 are also shown as having an exemplary rectangular shape. Each element 110 is about 4 mm long by about 0.5 mm wide with about 400 μm (or about 0.5 mm) between the elements 110 for a total span or array width of about 18 mm (or about 2 cm). These exemplary dimensions permit the capture of lateral beam profiles of a lithotripter on the length scale of the acoustic profile. The exemplary size dimensions of the elements 110 facilitate obtaining sufficient signal level and acceptable impedance. A suitable surface area for 50Ω impedance is about 6 mm².

The electrical connections 120 may be electrical trace lines as shown in FIG. 1 or microvias, which use holes to conduct electricity. The pads 130 are generally spaced at 1 mm increments and preferably fit into a standard zero-insertion force (ZIF) flex connector. The pads 130 correspond in number to the elements 100 and permit the array elements 110 to connect to the ZIF connector, which is a standard connection method for flex circuits. The pattern 100 is not limited to the dimensions mentioned above as the array pattern 100 may be made with any number of elements, in any range of sizes and shapes, and any spatial orientation of the elements for optimal performance in a given lithotripter field. Similarly, the array pattern 100 may be made with more complexity such as a cross array pattern or a two-dimensional array pattern.

Referring now to FIG. 3, an assembly of the present invention is shown where the polyimide film 140 etched with the pattern 100 is bonded to a piezopolymer membrane 160 with an epoxy 150. In a preferred embodiment, the piezopolymer membrane 160 is generally about a 9 μm film having about 80 MHz bandwidth. Exemplary piezopolymer membranes 160 include polyvinylidene fluoride (PVDF), P(Vdf-TrFE) or the like. P(Vdf-TrFE) has a higher coupling coefficient, kₜ, than PVDF, and thus generally makes for a better transducer material. A suitable example of a PVDF membrane is made by Ktech Corp. of Albuquerque, N. Mex. The piezopolymer membrane 160 has a first side and a second side, the second side may be electroded with a metal such as gold. The second side of the piezopolymer, or the metallized side, forms a ground plane of the transducer. The first side of the piezopolymer is bonded to the copper-clad polyimide (CCP) film with an epoxy.

As mentioned above, the assembly is fabricated using an epoxy 150, specifically a non-conductive epoxy, to bond the piezopolymer membrane 160 to the polyimide film 140. The films are then pressed together during the bonding process to form the assembly and finally cured. Generally, a small amount of the non-conductive epoxy 150 is placed on a first side of the polyimide film 140 after which the first side of the piezopolymer membrane 160 is placed on the epoxy. The epoxy may include any resinous bonding agent, a suitable epoxy includes Hysol RE2039 or HD3561, manufactured by Loctite Corp. of Olean, N.Y.

The linear hydrophone array transducer device of the present invention may be fabricated in many forms. In one embodiment, the piezopolymer membrane 160 and polyimide film 140 may be bonded together with the epoxy 150 by pressing the membrane 160 and film 140 between flat metal plates. The flat plates may be two aluminum plates used to press the membrane 160 and the film 140 together into a planar geometry.

In another embodiment, the array transducer device may be fabricated by pressing into a metal mold. Here, the membrane 160, the film 140 and the epoxy 150 between the two are pressed into the mold so that the epoxy 150 may bond the piezopolymer membrane 160 and the polyimide film 140 together. The mold may take the form of a metal cylinder or sphere thus permitting the device to have a curved surface. Generally, any desired curvature may be attained by choosing a suitable mold geometry. To attain more complicated curvatures, it may be advantageous to form the final array transducer device from several sub-units.

FIG. 2 shows yet another embodiment of the linear hydrophone array transducer device 200. Here, the membrane 160 and the film 140 may be first bonded by pressing between flat metal plates to achieve a planar geometry, cured, and then attached to a Teflon mold being about 15 mm deep. The mold may be secured to the second side or backside of the polyimide film 140 directly over the array pattern 100 using a sealant such as RTV sealant, or the like. The mold may be a rectangular piece of Teflon with a slot running through it and with an interior that may be back-filled with additional epoxy. If the array transducer device 200 uses microvias as electrical connections, then wires must be bonded to the elements before backfill material is inserted. Once the epoxy cures, the device will be separated from the mold and excess material trimmed away leaving the device 200.

The transducer device 200 is shown bonded to the epoxy backing plug 220 (See FIGS. 2 and 3) with a flap for the electrical connections. The epoxy backing plug 220 may serve to secure the geometry of the array pattern 100 and may provide a means to mount the transducer onto a bracket or other devices. For example, a threaded shaft or a specialized connector may be embedded in the epoxy backing plug 220 before it cures. The array transducer device 200 may be fabricated in any of the above-mentioned ways. In any of the described fabrication methods, generally, about a 1 μm thick epoxy layer 150 remains after curing the film 140 and membrane 160.

In an alternative embodiment of the present invention, a thin protective coating may be applied on the device 200. Suitable coatings include Mylar, silicone or polypropylene membranes being about 50 μm. The coating may be useful in reducing the risk of cavitation damage to the piezopolymer membrane 160 of the device 200. The coating shifts the cavitation damage to the replaceable membrane.

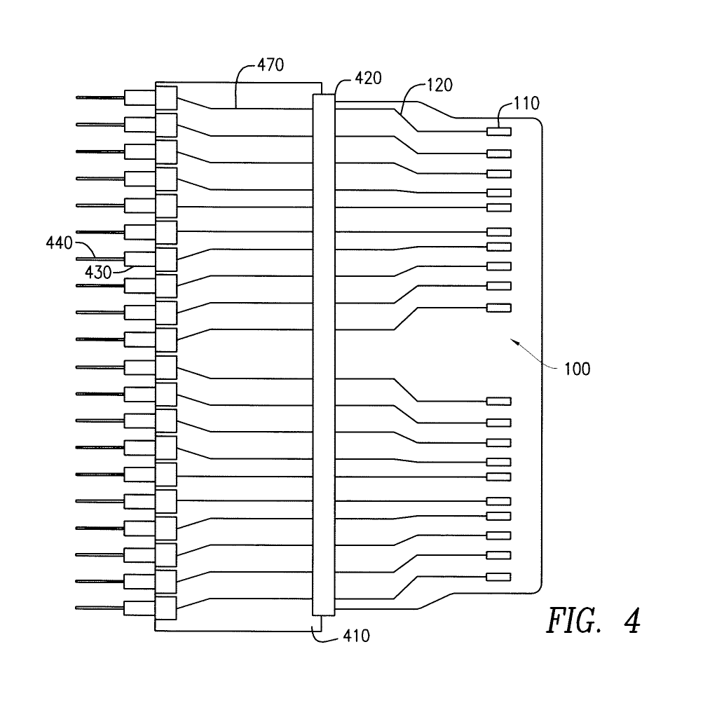

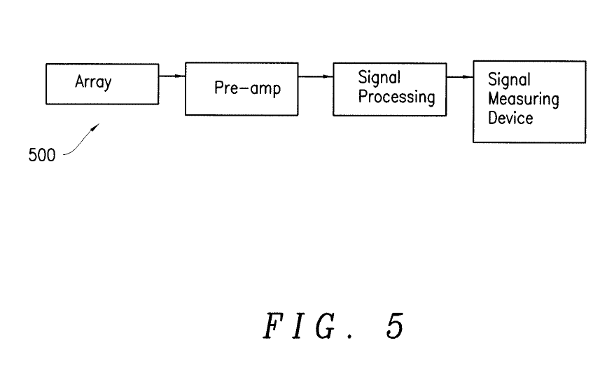

Referring now to FIG. 4. A customized printed circuit board (PCB) 410 can be fabricated to enable electronic access to the array pattern 100 through the printed circuit board traces 470. The PCB 410 has a connector 420 on one side and a series of smaller connectors 430 on the opposing side. Cables 440 are connected to each of the smaller connectors 430. In a preferred embodiment, there are twenty smaller connectors 430. A mounting bracket made from an aluminum rod may be used to hold the transducer 200 and the PCB 410. Thus, the PCB 410 enables electronic access from the cables 440 to the PCB traces 470 through the connectors 430. The PCB traces 470 are electronically connected to the electrical connections 120 of the transducer device 200 through a connector 420. The electrical connections 120 are electronically connected to the elements 110.

In a preferred embodiment, the first connector 420 is a 20-pin zero insertion force (ZIF) connector, which is commercially available from Hirose Electric located in Simi Valley, Calif. or any equivalent supplier. The smaller connectors 430 are miniature MMCX-BNC connectors, which are commercially available from Amphenol or any equivalent supplier. The PCB 410 links the ZIF connectors 420 to the BNC connectors 430. The cables 440 are BNC cables, such as RG-174 50-Ω of 0.87 meters length.

The PCB 410 is designed to permit the placement of optional high-input-impedance pre-amps and optional surface mount inductors. The pre-amps provide a 50-Ω output impedance. The pre-amps may be disposed close to the array elements 110 to help minimize frequency-dependent transmission-line losses. Preferably, low-gain (2×), high bandwidth about 100 MHz pre-amps should be utilized in the present invention. Suitable commercially available pre-amps include AD8334, manufactured by Analog Devices of Norwood, Mass. In some embodiments, the optional surface mounted inductors may be soldered directly onto the PCB 410 to perform impedance matching.

FIG. 5 shows a system configuration 500 between the array transducer device 200 and a signal measuring device. Preamplification may optionally be employed to provide a small amount of analog gain and also to have a defined output impedance. Signal processing can consist of filters, peak detectors, or other analog modifications of the signal. Suitable examples of signal measuring devices include signal digitizing devices such as a digital oscilloscope or similar devices. It should be noted that the configuration 500 is provided for illustrative purposes and should not be construed to be limiting the present invention in any way.

The linear array transducer device of the present invention is suitable for single-shot measurements of a pressure or force field such as an acoustic field of a shock wave lithotripter. Measurement can be made for waveform, temporal peak, acoustic beamwidth, beamwidth variability, peak pressures and shock duration. The transducer device is advantageous in being able to make instantaneous pressure measurements at many points simultaneously rather than making a point-by-point measurement. The device is further advantageous in being able to take measurements regardless of shock variability.

The following experiments measuring a lithotripter acoustic field further illustrate the present invention, but should not be construed as in any way limiting its scope.

Experiments

SWL measurements were made in degassed water with the linear hydrophone array transducer device of the present invention placed at the focus of a research lithotripter modeled after the Dornier HM3. The hydrophone array was oriented at the focus such that the area of each element was approximately perpendicular to the axis of propagation of the shock wave. Moreover, the array was centered such that the middle elements were aligned at the geometric focus of the lithotripter.

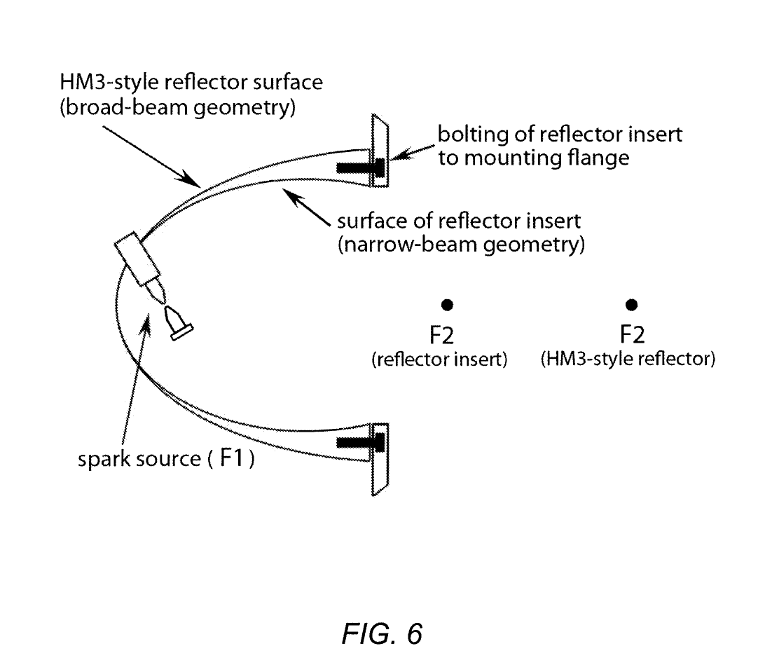

A charging potential of 15, 18, or 23 kV was used to trigger single shock waves at a rate slower than one per minute. In the experiment, two ellipsoidal reflectors were used: an HM3-style reflector and a reflector insert that fits inside the HM3-style reflector. See FIG. 6. The ellipsoidal reflectors possess two foci; F1 denotes the focus corresponding to the spark source and F2 denotes the remote focus corresponding to the treatment site and the central location at which beamwidth measurements were acquired. The HM3-style reflector had semi-major and semi-minor axes: a=13.80 cm, b=7.75 cm while the reflector insert had a=9.30, b=6.24. Both reflectors were axisymmetric in that they did not possess cutouts to accommodate fluoroscopy that typically exist in clinical lithotripters. The reflector insert was designed to create a tighter focus, as illustrated in FIG. 6. New electrodes (<200 shock waves) and worn electrodes at the end of their prescribed clinical lifetime (>2000 shock waves) were used. New electrodes generally produced more reproducible sparks, while worn electrodes possessed larger spark gaps and resulted in more variable sparks. Testing with both new and worn electrodes ensured variability in the focal pressures.

Voltage measurements from the array elements were captured on six digital oscilloscopes with sampling rates of at least 50 MHz. Twenty elements were connected to the inputs of the six digital oscilloscopes via BNC cables and the measurements were collected using an input impedance of 1 MΩ on the oscilloscopes without preamplification. A custom LabVIEW program (National Instruments, Austin, Tex.) was used to digitally store select waveforms. Oscilloscope measurements corresponding to peak positive pressures of the focal wave were manually recorded for all array elements. For select shock waves, oscilloscope settings were adjusted to resolve the peaks corresponding to the direct wave (i.e. the spherically diverging wave generated by the spark that precedes the focal wave).

Measurements were analyzed in two steps. First, given that the direct wave diverges spherically from the spark at one focus of the ellipsoidal reflector, the relative amplitude of the direct wave as experienced by each array element was calculated from geometric considerations. Next, the direct-wave measurements were used to normalize measurements of the focal wave on an element-by-element basis. In this manner, variations in the sensitivity of each element were accounted for thereby enabling quantitative measurement of the relative pressure amplitude of individual shock waves across the focal width of the lithotripter. Although not measured explicitly in this effort, absolute pressures for the Dornier HM3 reflector have been measured previously. For this geometry, the maximum peak positive amplitudes have been reported to be about 1 MPa for the direct wave and 29.9±4.7 MPa for the focused wave. The ratio of these reported amplitudes can be directly compared to the data presented below for the HM3-style reflector.

Results

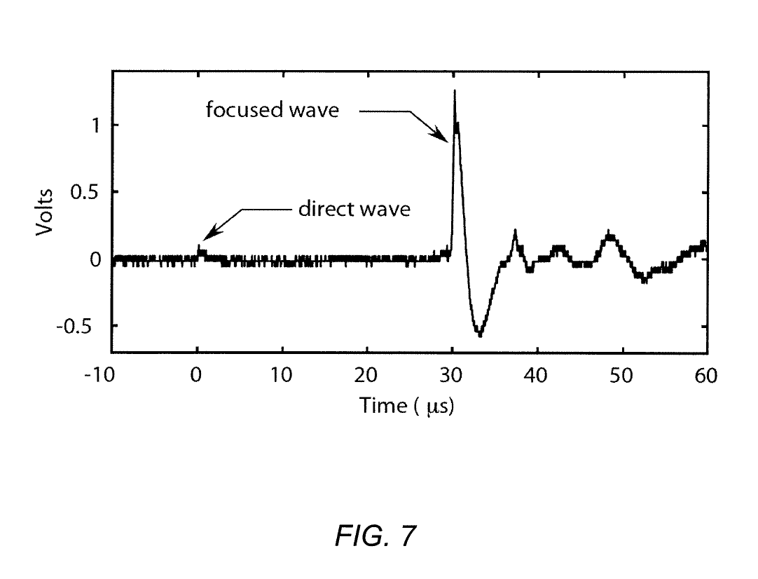

An example of a waveform measured on a single element of the array hydrophone while using the HM3-style reflector and a new electrode is shown in FIG. 7. The sensitivity of the hydrophone array was sufficient to reveal the direct wave seen at t=0 as well as the focused wave at t≈30 μs. The measured focal waveforms show a peak positive spike of about 1-μs duration followed by a negative trough of about 4 μs, which is a commonly described classic lithotripter waveform.

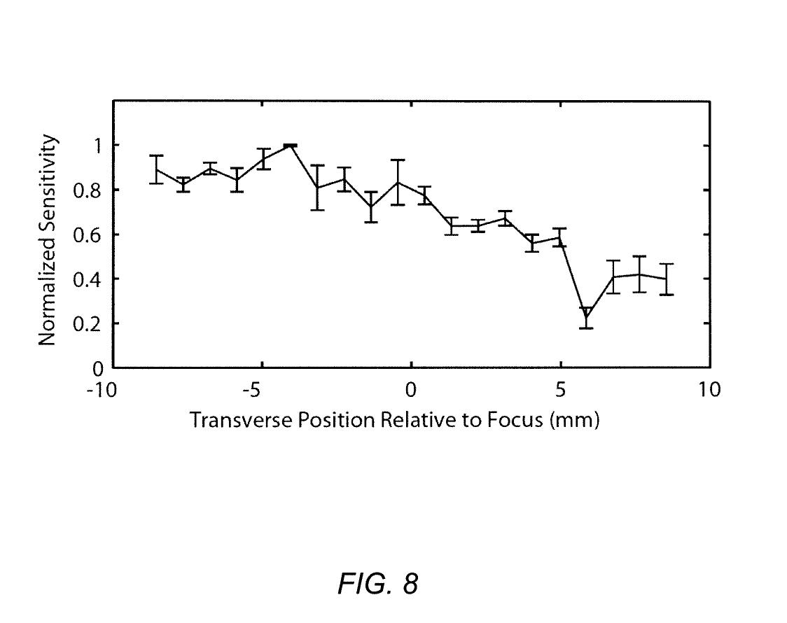

In order to resolve the direct waveforms across the array elements, alternate oscilloscope settings were used to acquire data at 18 and 23 kV charging potentials for the HM3-style reflector and at 15 and 18 kV for the reflector insert. To reduce the risk of damage to the most sensitive element, direct-wave measurements with the HM3-style reflector were normalized relative to the most sensitive element for each shock wave. These normalized responses for six separate shock waves were then averaged. Referring to FIG. 8, the thick line shown indicates the element-by-element measurements from six shock waves, while the vertical bars indicate ±1 standard deviation at each element. As illustrated in FIG. 8, element sensitivities tended to decrease across the width of the array. The element sensitivities reported in FIG. 8 were repeatably observed.

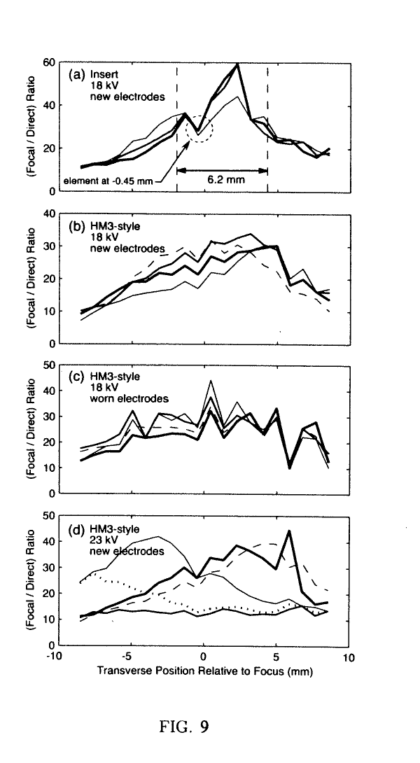

After correcting for individual element sensitivities as described above, the beam profiles of individual shock waves are presented in FIG. 9. Shock waves were generated as follows: FIG. 9(a) shows the reflector insert, a charging potential of 18 kV, emission of 3 shock waves, and use of new electrodes; FIG. 9(b) shows the HM3-style reflector, a charging potential of 18 kV, emission of 4 shock waves, and use of new electrodes; FIG. 9(c) shows the HM3-style reflector, a charging potential of 18 kV, emission of 4 shock waves, and use of worn electrodes; and FIG. 9(d) shows the HM3-style reflector, a charging potential of 23 kV, emission of 5 shock waves, and use of new electrodes. The −6 dB beamwidth of the thickest line profile is marked in FIG. 9(a).

Because all measurements are normalized relative to the direct wave from the HM3-style reflector at 18 kV, the plotted amplitudes can be directly compared. As shown, measured profiles are sorted based on the test conditions used to generate shock waves. Focused beams were produced using four distinct combinations of either the HM3-style or the insert reflector, a charging potential of 18 kV or 23 kV, and new or worn electrodes. In FIG. 9(a), the −6-dB beamwidth of the profile corresponding to the thickest line is explicitly marked and labeled. An array element at a transverse position of −0.45 mm (as labeled by the dashed circle) consistently measured lower pressures than its neighbors in all profiles. Accordingly, this element was ignored in the estimation of beamwidths.

Each of the profiles plotted in FIG. 9 was used to estimate the −6-dB beamwidth and the transverse location of the peak pressure. These data for independently measured shock waves are provided in Table I, in which “Range” denotes the extent of the range of measurements as the difference between the maximum and minimum values. Comparing the four separate groups of shock-wave measurements, several key differences are apparent. First, from test conditions in FIG. 9(a) and FIG. 9(b), the insert reflector produced narrower beamwidths, higher peak pressures, and a more consistent localization of the peak pressure than did the HM3-style reflector. In Table I, the range of zero for peak pressure locations indicates that the peak was measured at the same element for all shock waves. Moreover, despite measuring a low number of shock waves, the beamwidths measured for conditions FIG. 9 (a) and FIG. 9 (b) are statistically different at a 95% confidence level (p≈0.04). Considering conditions FIGS. 9 (b), (c), and (d) for the HM3-style reflector, worn electrodes altered the shape of the beam profiles and generated slightly higher peak pressures than new electrodes at a charging potential of 18 kV.

TABLE I
Measurements from individual shock waves.

| | | Beamwidth | | Peak Location |
|---|---|---|---|---|
| | | (mm) | | (mm) |
| Conditions | | Mean | Range | Range |
| (a) | Insert reflector | 8.1 | 6.3 | 0.0 |
| | 18 kV, new electrodes | | | |
| | [3 shock waves] | | | |
| (b) | HM3-style reflector | 13.1 | 2.6 | 5.4 |
| | 18 kV, new electrodes | | | |
| | [4 shock waves] | | | |
| (c) | HM3-style reflector | 11.9 | 4.5 | 5.4 |
| | 18 kV, worn electrodes | | | |
| | [4 shock waves] | | | |
| (d) | HM3-style reflector | 12.4 | 6.9 | 14.4 |
| | 23 kV, new electrodes | | | |
| | [5 shock waves] | | | |

At a charging potential of 23 kV with new electrodes, the HM3-style reflector tended to produce higher peak pressures than at 18 kV, as expected. However, shock waves generated at 23 kV also exhibited much greater variability as indicated by the plotted profiles as well as by the reported 14.4-mm range for peak pressure locations. Assuming that the measured locations of peak pressure are normally distributed, the variances of the distributions corresponding to conditions FIG. 9 (b) and FIG. 9 (d) are different with a statistical confidence level of 89%. This result again demonstrates that the array can be used to discern system variability based on only a few shock wave measurements. In this case, higher charging potential affected the repeatability of the location of peak pressure.

In addition to the above comparisons among test conditions used in this effort, measurements with the HM3-style reflector can also be compared to data derived from single-point hydrophone measurements. The −6-dB beamwidth of the HM3-style reflector is about 14 mm. Under all conditions tested in this effort, the beamwidths were 1-2 mm less than the previously reported value for the HM3-style reflector. Although this discrepancy may not be statistically significant, these observations are consistent with the expectation that averaging of single-point measurements from multiple shock waves leads to an overestimation of beamwidth. Aside from beamwidth, testing was performed using a single-point hydrophone to measure peak positive pressures of 29.9±4.7 MPa at 18 kV for the device used in this effort. Assuming a direct-wave amplitude of 1 MPa as mentioned above, the expected range of normalized peak pressures from 25-35 is confirmed by the profiles in FIG. 9(b).

The primary focus of this effort was to explore the use of a novel hydrophone array for single-shot measurements of lithotripter shock waves. One particular use of this array in lithotripsy research is to enable quick and accurate comparisons of the beamwidths produced by various reflector geometries.

### CONCLUSIONS

The linear hydrophone array of the present invention is a useful tool for quickly and accurately measuring the beamwidths of focused shock waves. The measurements presented above represent the first simultaneous acoustic field measurements in an electrohydraulic lithotripter. Previous measurements consisted of point measurements at a single spatial location for each shock wave. Using 20 elements, the beam profiles measured with the array were relatively smooth. Although some variabilities persisted after compensation for the sensitivities of individual elements, the instantaneous measurements did enable at least a qualitative distinction of the acoustic fields generated under different test conditions. Notably, the beamwidths of the HM3-style and the insert reflectors were determined to be statistically different after acquiring measurements from only a few shock waves.

While the invention has been described by way of example and in terms of specific embodiments it is not so limited and is intended to cover various modifications as would be apparent to those skilled in this art area. Therefore, the scope of the appended claims should be accorded the broadest interpretation so as to encompass all such modifications.

1. A method for measuring acoustic pressure at multiple locations simultaneously comprising:
providing a transducer having a polyimide film bonded to a piezopolymer membrane, wherein the film is etched with a linear array pattern, the array pattern having at least two array elements;
enabling access to the array pattern by a detection device;
placing the transducer in the field of a shock wave emitted by a lithotripter; and
acquiring acoustic field pressure data at multiple locations simultaneously from the emitted shock wave with said detection device.

2. The method of claim 1, wherein the transducer is placed at the focus of the lithotripter.

3. The method of claim 1, wherein each array element is approximately perpendicular to an axis of shock wave propagation.

4. The method of claim 1, wherein the array pattern is centered such that elements in the middle of the array pattern are aligned at the geometric focus of the lithotripter.

5. The method of claim 1, wherein voltage measurements are collected by said detection device.

6. A method of fabricating a device for measuring acoustic pressure at multiple locations simultaneously comprising:
providing a piezopolymer membrane, and
providing a polyimide film bonded to the piezopolymer membrane, the membrane and the film being pressed into an assembly, said film further comprising a linear array pattern etched into the film, the array pattern having a plurality of array elements, connecting pads and electrical connections connecting the pads to the array elements;
wherein the assembly when placed in an acoustic pressure field obtains signal data at multiple locations from a single shock wave in any dimension.

7. The method of claim 6, wherein the polyimide is a copper-clad polyimide film having a first side and a second side, wherein bonding said film to said membrane is with a non-conductive epoxy that is deposited on the first side of the film.

8. The method of claim 6, wherein the piezopolymer membrane has a first side and a second side, said first side of the piezopolymer membrane being placed on the epoxy.

9. The method of claim 3, wherein the second side of the piezopolymer membrane is coated with a replaceable membrane.

10. The method of claim 6, wherein the assembly is created by press fitting the membrane and the film between two metal plates to achieve a planar geometry.

11. The method of claim 6, wherein the electrical connections are microvias.

12. The system of claim 6, wherein the electrical connections are electrical trace lines.

13. The method of claim 6, wherein the array pattern is one dimensional.

14. The method of claim 6, wherein the array pattern is two dimensional.

15. The method of claim 6, wherein the array pattern is in a cross pattern.

16. The method of claim 6, wherein there are 20 elements that are equally spaced apart.

17. The method of claim 6, wherein the elements span about 18 mm.

18. The method of claim 6, wherein the pads fit into a standard zero-insertion force flex connector.

19. The method of claim 6, further comprising a printed circuit board enabling electronic access to the array pattern, the printed circuit board having traces, wherein the traces are electronically connected to the electrical connections of the array pattern.

20. The method of claim 6, wherein there is about 400 μm between the element.
